(19) United States
(12) Patent Application Publication
(10) Pub. No.: US 2023/0098979 A1
(43) Pub. Date: Mar. 30, 2023

(54) METHODS AND SYSTEMS FOR RELEASING A PILL

(71) Applicant: Incaveo, Inc., San Francisco, CA (US)

(72) Inventors: Andrew Cohen, San Francisco, CA (US); Hoyman Hong, Burlingame, CA (US)

(73) Assignee: Incaveo, Inc., San Francisco, CA (US)

(21) Appl. No.: 17/800,099

(22) PCT Filed: Feb. 18, 2021
(86) PCT No.: PCT/US2021/018581

§ 371 Date: Aug. 16, 2022

Related U.S. Application Data
(60) Provisional application No. 62/978,725, filed on Feb. 19, 2020.

(51) Int. Cl. A61J 1/03; A61J 7/04

(52) U.S. Cl. CPC A61J 1/035; A61J 7/0481; A61J 7/0418

(57) ABSTRACT

The present disclosure provides methods and systems for managing medications by releasing them on a controlled access or schedule. The methods and systems may comprise the use of a pill release device. The pill release device may comprise a substrate comprising one or more containers, a cover configured to rest adjacent to one or more walls of the container, a fluid source in fluid communication with the cover, and circuitry operably coupled to the fluid source. The circuitry may send one or more electrical signals which may cause the fluid source to move a fluid to or away from the cover. The movement of the fluid may seal or unseal the over against the one or more walls, which may permit the pill to be received by or dispensed from the container.

### PRIORITY CLAIM

This application claims priority to U.S. Provisional Patent Application No. 62/978,725, filed Feb. 19, 2020, which is incorporated by reference in its entirety.

### FIELD OF THE INVENTION

This application relates to methods and systems for releasing a pill.

### BACKGROUND

Drug overdoses can be accidental or intentional. Drug overdose may occur when a person takes more than the medically recommended dose. Drug overdose can cause serious health and societal problems. For example, the opioid epidemic has become a public health emergency in the United States with approximately 130 mortalities due to overdose every day and over $78.5 billion lost every year due to lost productivity, healthcare, and addiction treatment. Despite risks of addiction with opioid treatment, patients may still be prescribed opioid pain medication in quantities often larger than necessary. Given the frequency of common but painful outpatient surgeries, it may be important to gain adequate pain control without the risk of addiction.

### SUMMARY

The opioid epidemic has become a public health emergency affecting the United States with over 400,000 lives lost from 1999 to 2017 with 47,600 fatal overdoses in 2017 alone. Approximately 130 people die from opioid overdoses every day, the majority of these individuals between the ages of 25-55. The economic toll due to this opioid epidemic can be severe with the morbidity and mortality between 2015-2018 accounting for over $250 billion lost due to lost wages, an estimated $96 billion loss due to lost productivity, and over $205 billion spent on healthcare for those afflicted with opioid addiction and their families.

Despite the known risks of addiction with opioids, patients may still be regularly expected to self-manage their pain and prescribed opioid pain medications by their family physician or after an outpatient surgery in quantities larger than necessary. This may create an environment conducive to addiction by allowing patients to take too many pills too often, take medication for too many days after their pain has subsided, and may allow excess medication to be used for non-medical purposes or diverted to a family member, fiend, or coworker to illicitly use or sell.

The probability a patient uses opioids one year after surgery may be related to the number of days they used opioids post-operatively. Some patients who ceased opioid use within three days post-surgery had less than a 5% probability of using opioids, while patients using opioids five days post-surgery had a 10% risk of opioid use one year later, and patients using opioids twenty days later had a 35% risk. These findings may be significant given that over 2 million patients every year in the United States have common but painful outpatient surgeries such as knee arthroscopy, mastectomy, cholecystectomy, and hernia repairs and may be prescribed an opioid medication for the first time. Hill, M. V., et. al. looked at opioid prescription rates and reported opioid usage of patients who undergo these common and painful outpatient surgeries and saw post-surgical opioid prescriptions may vary from a few to thirty days and prescriptions were over 70% larger than the patients' reported use. Additional studies looking at prescription practices for knee arthroscopy showed that for adolescents prescriptions may be over 67% larger than reported usage and over 84% for adults. Patients with larger prescriptions may use more medication increasing their risk of addiction.

Standardization of opioid prescriptions for common surgeries have been proposed and both multiple states as well as the FDA recommending limitations on the quantity of pain medications that may be prescribed. However, surgeons may be unaware of standardized prescription guidelines and physicians may believe state and FDA controls are overly restrictive and not representative of patient needs post-surgery.

Currently there is a need for methods or technologies that support the clinical desire to provide pain relief for the shortest times and dosages possible while recognizing the wide variation in pain reported by patients without giving access to extra medication. Smart pill dispensers may aid medication compliance, but these devices may not control access to opioids as patients generally fill these devices with their prescriptions manually, which may defeat any benefit of controlled access. Other types of pill dispensers may be limited by their logistics in that they may require all medication to be delivered in secured cartridges, requiring changes to the fulfillment process and supply chain which may be difficult to implement. They can also place an additional burden on post-surgical patients due to the at home usage. It may be important to ensure patients have access to opioids when needed, the minimum time intervals between doses and the maximum number of days can be enforced, and there may be deterrents to dissuade patients from using opioids past the expected usage for a particular surgery.

Provided herein are systems and methods for managing medications by providing their controlled access or scheduled release. The systems may comprise a pill release device. The pill release device may provide a controlled access for one or more medications in a pre-measured dose at the appropriate time for the patient to take the medication. The pill release device may comprise one or more sensors which may monitor the release of the pills. A sensor may be incorporated into the blister or a blister pack and detect if a pill is present or absent in the blister. A sensor may be incorporated into the backing of a blister pack and may detect if the backing is removed. The sensor may be in communication with or may be incorporated into an electronic unit (e.g., circuitry) in the device, which electronic device may send electrical signals to lock the device, restricting access to the pills upon occurrence of certain event(s), e.g., after a predetermined number of pills have been released. The pill release device may comprise no moving parts. In some aspects, the present disclosure provides a device for releasing a pill comprising of a substrate comprising a container, which container comprises one or more walls and an opening configured to permit a pill to be received by or dispensed from the container, a cover configured to rest adjacent to the one or more walls, wherein the cover and container are configured to undergo motion relative to one another, a fluid source in fluid communication with the cover, wherein the fluid source is configured to supply a fluid to at least a portion of the cover, and wherein an application of the fluid from the fluid source to the portion of the cover seals the cover against one or more walls, and circuitry operably coupled to the fluid source, wherein the circuitry is configured to supply one or more electrical signals to cause the fluid source to direct the fluid to or away from the portion of the cover, which one or more electrical signals are sufficient to permit the cover and one or more walls to undergo motion relative to one another, to thereby permit the pill to be received by or dispensed from the container. In some embodiments, the substrate comprises a plurality of containers. In some embodiments, the device is a blister pack. In some embodiments, the plurality of containers are blisters in a blister pack. In some embodiments, the cover is an adhesive pad. In some embodiments, the cover has a multi-layer structure. In some embodiments, said cover comprises said fluid source. In some embodiments, said cover has a thickness of less than or equal to about 5 millimeters. In some embodiments, said fluid comprises a polar compound. In some embodiments, said circuitry is embedded in said substrate. In some embodiments, said circuitry is an integrated circuit chip. In some embodiments, said circuitry is a mini-chip. In some embodiments, said cover is a switchable electronic capillary adhesive device (SECAD). In some embodiments, said SECAD comprises a top layer having opposing inner and outer major surfaces, at least two fluidic thru-passageways, and a sealable fluid holder. In some embodiments, said fluidic thru-passageways comprise a plurality of fluidic thru-passageways. In some embodiments, said portion of said cover comprises one or more fluidic thru-passageways. In some embodiments, said SECAD comprises at least one electro-osmotic pump operatively coupled to said fluid source. In some embodiments, said electro-osmotic pump comprises a plurality of electro-osmotic pumps. In some embodiments, said electro-osmotic pump is operably coupled to a battery. In some embodiments, said electro-osmotic pump is operably coupled to an electrode pair. In some embodiments, said electrode pair comprises a top electrode and a bottom electrode. In some embodiments, said circuitry is further configured to store information to direct a sequence of sealing and unsealing events. In some embodiments, said one or more electrical signals direct sealing of said cover against said one or more walls for a predetermined time period. In some embodiments, said predetermined time period is greater than or equal to 4 hours. In some embodiments, further comprising a sensor operably coupled to said circuitry. In some embodiments, said sensor is comprised in said container. In some embodiments, said sensor sends one or more signals to said circuitry, which one or more signals indicate when a pill is removed from said container. In some embodiments, said sensor is an IR sensor paired with an IR transmitter. In some embodiments, said sensor is a pressure sensor. In some embodiments, said sensor is a proximity sensor. In some embodiments, said sensor is a light sensor. In some embodiments, said circuitry is further configured to track inventory of pills within said device. In some embodiments, further comprising a communications unit which communicates to an external device. In some embodiments, said communications device is a near-field communications device. In some embodiments, said communications device is cellular modem. In some embodiments, said communications device is RFID. In some embodiments, said communications device is a Bluetooth transmitter. In some embodiments, said communications device is a USB. In some embodiments, said communications device is a WIFI transmitter. In some embodiments, said communications device is a WIMAX transmitter. In some embodiments, said communications device is an Ethernet connection. In some embodiments, said communications device communicates to said external device through a mobile application. In some embodiments, said apparatus comprises no moving parts.

In some aspects, the present disclosure provides a method for receiving or releasing a pill, comprising providing a device comprising: a substrate comprising a container, which container comprises one or more walls and an opening configured to permit said pill to be received by or dispensed from said container; a cover configured to rest adjacent to said one or more walls, wherein said cover and said container are configured to undergo motion relative one another; a fluid source in fluid communication with said cover, wherein said fluid source is configured to supply a fluid to at least a portion of said cover, and wherein an application of said fluid from said fluid source to said at least said portion of said cover seals said cover against said one or more walls; and circuitry operably coupled to said fluid source, wherein said circuitry is configured to supply one or more electrical signals to cause said fluid source to direct said fluid to or away from said at least said portion of said cover, which one or more electrical signals are sufficient to permit said cover and said one or more walls to undergo motion relative to one another, to thereby permit said pill to be received by or dispensed from said container; and using said circuitry to supply said one or more electrical signals to said fluid source to direct said fluid to or away from said at least said portion of said cover, thereby causing said cover and said one or more walls to undergo motion relative to one another to permit said pill to be received by or dispensed from said container. In some embodiments, said cover has a multi-layer structure. In some embodiments, said cover comprises at least one electro-osmotic pump operably coupled to said fluid source. In some embodiments, said application of said fluid from said fluid source unseals said cover against said one or more walls. In some embodiments, said circuitry is part of the cover. In some embodiments, said circuitry is further configured to store information to direct a sequence of sealing and unsealing events. In some embodiments, further comprising directing said sealing of said cover against said one or more walls for a predetermined time period using said one or more electrical signals. In some embodiments, the device further comprises a sensor. In some embodiments, further comprising using the sensor to detect whether said pill is removed from or dispensed into said container. In some embodiments, further comprising using said sensor to record a time when said pill is removed from or dispensed into said container. In some embodiments, said circuitry is further configured to supply said one or more electrical signals within a predetermined time period. In some embodiments, said predetermined time period is less than or equal to about 7 days. In some embodiments, further comprising locking said device after a predetermined time period during which said pill is not removed from said container. In some embodiments, said predetermined time period is greater than or equal to 24 hours. In some embodiments, said device comprises a plurality of containers each comprising at least one pill. In some embodiments, said one or more electrical signals permit said plurality of containers to undergo a sequence of locking and unlocking events. In some embodiments, said locking and unlocking events occur at a given time interval. In some embodiments, said time interval is greater than or equal to about 4 hours. In some embodiments, the method further comprises locking said device after a given percentage of said pills are removed from said device. In some embodiments, said given percentage is at least about 50%. In some embodiments, said given percentage is at least about 80%. In some embodiments, said circuitry is further configured to execute scripts to direct said locking and unlocking events, set said time interval of said locking and unlocking events. In some embodiments, said circuitry is in communication with an external device to accept an input comprising said scripts.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”and “FIG.”herein), of which:

FIG. 1 illustrates a schematic of the use of an example device for receiving or releasing a pill.

FIG. 2 illustrates a cut-away view schematic of a Switchable Electronically-controlled Capillary Adhesion Device (SECAD).

FIG. 3 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the term “capillary adhesion” generally refers to the surface tension force from a large number of small liquid bridges in parallel. A fluid mechanics-based bond using surface tension may comprise a liquid surface tension acting along the perimeter of a wetted contact area to give a force for a single contact dependent on the diameter of contact with the surface such as a liquid droplet caught between two glass histology slides.

As used herein, the term “electro-osmosis” generally refers to the motion of liquid induced by an applied potential across a porous material, capillary tube, membrane, or any fluid conduit. The Coulomb force induced by an electric field on net mobile charge in a solution induces electro-osmotic flow. The chemical equilibrium between a solid surface and an electrolyte solution typically leads to the interface acquiring a net fixed electrical charge, a layer of mobile ions forms in the region near the interface. When an electric field is applied to the fluid, the net charge in the electrical double layer is induced to move by the resulting Coulomb force. An electro-osmotic switch can exploit these mechanisms to create a bi-stable system of liquid-gas interfaces with a trigger to toggle back and forth between the energy wells. Such a switch may consist of a pair of coupled droplets whose shape-change is triggered by volume transfer using an electro-osmotic pump placed between the two droplets.

As used herein, the term “sensor” generally refers to a device whose purpose is to detect events or changes to its environment and send the information to coupled electronics. A sensor may be an electric current or potential sensor, a pressure sensor, a proximity sensor, an optical sensor, or any sensor that is able to differentiate an object from its environment. In some embodiments described herein, a sensor may be a device, module, or subsystem capable of sensing the presence or absence of a pill within a pill release device.

As used herein, the term “electronic control” generally refers to an embedded circuit system that controls one or more of the electrical systems or subsystems in a device. An electronic control may transform one signal into another so as to give a desired system response. In a pill release device, an electronic control may switch a battery on or off, direct a series of locking or unlocking events to release a pill, control a pump, or provide external communications with an integrated device or application.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” is used to indicate that a value includes the inherent variation of error for the device, the method being employed to determine the value, or the variation that exists among the study subjects. Unless otherwise specified based upon the above values, the term “about” means ±5% of the listed value.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

An aspect of the present disclosure provides systems of methods for releasing a prescription drug that ensures prescription may be taken only as directed, not diverted, and/or for the minimum number of days thus achieving adequate pain control while minimizing risk of addiction.

In some cases, systems of the present disclosure may comprise a device capable of receiving or releasing a pill. The device may comprise a substrate which may comprise a container. The container may comprise one or more walls and an opening configured to permit the pill to be received by or dispensed from the container. The container may further comprise a cover configured to rest adjacent to these one or more walls where the cover and container are configured to undergo motion relative one another. Additionally, the device may comprise a fluid source in fluid communication with the cover. The fluid source may be configured to supply a fluid to at least a portion of the cover, and where an application of the fluid from the fluid source to the portion of the cover may seal the cover against one or more walls. In some cases, the device further comprises circuitry operably coupled to the fluid source, where the circuitry may be configured to supply one or more electrical signals which may cause the fluid source to direct the fluid to or away from the portion of the cover. The one or more electrical signals may be sufficient to permit the cover and the one or more walls to undergo motion relative to one another, and thereby may permit the pill to be received by or dispensed from the container.

In some cases, the substrate comprises a plurality of containers (e.g., greater than or equal to about 2, 3, 4, 5, 6, 7, 8, 9, 10, 12, 14, 16, 18, 20, or more containers). The container may be a cavity, a pocket, a hole or the like. The container may take different shapes, regular or irregular. A potential configuration of this substrate may comprise one or a plurality of containers such that each prescribed pill is housed inside a container. The substrate may be a bottle, a blister pack, or some configuration thereof. The blister pack may be a clamshell container. The plurality of containers may be blisters in a blister pack. The blister pack may be a pre-formed packaging used for small consumer goods and pharmaceuticals. The blister pack may protect products against external factors such as humidity or UV light which may diminish the efficacy of the product within or may be used to keep the product locked securely until an appropriate access time such as in the release of a pharmaceutical compound. The blister pack may comprise a cavity or pocket to house a product, such as a pill, that is backed by a lidding seal of aluminum foil, plastic, or other malleable metal such that the backing material may be ruptured allowing access to the cavity and its contents.

The blister may be a chamber made out of some variety of plastic or other suitable material. The chamber can be translucent. A blister may be but is not limited to a formed material such as polyvinyl chloride, polyvinylidene chloride, polypropylene, polyethylene, polychlorotrifluoroethylene, cyclic olefin polymers, aluminum, polyamide, or cold form foil. The formed plastic may be formed through thermoforming, cold forming, chemical extrusion processes, or mechanical processes among others.

The cover configured to rest adjacent to the walls of the containers may be an adhesive pad. Such an adhesive pad may provide a locking mechanism. The cover may have a multi-layer structure (e.g., greater than or equal to 2, 3, 4, 5, or more layers). The cover may have a thickness of less than or equal to about 5 millimeters (mm). The cover may have a thickness greater than or equal to about 0.5 mm, 1 mm, 1.5 mm, 2 mm, 2.5 mm, 3 mm, 3.5 mm, 4 mm, 4.5 mm, 5 mm, or more. The cover may have a thickness less than or equal to about 5 mm, 4.5 mm, 4 mm, 3.5 mm, 3 mm, 2.5 mm, 2 mm, 1.5 mm, 1 mm, 0.5 mm, or less. The cover may have a thickness falling between any of the two values described above, for example, about 3 mm.

The fluid source may be internal or external to the cover. The fluid supplied by the fluid source may be a polar compound. A polar compound may be water (e.g., distilled water), methanol, ethanol, acetone, dichloromethane, ethyl acetate, butanol, propanol, tetrahydrofuran, acetonitrile, dimethylformamide, dimethyl sulfoxide, or ammonia. The fluid source may be a non-polar liquid (i.e., organics as opposed to water) when properly doped, thus having an ‘effective’ zeta potential. A non-polar liquid may be toluene, chloroform, pentane, octane, hexane, cyclohexane, isooctane, trimethylpentane, heptane, xylene, or benzene. A non-polar liquid may be doped with a charge control agent such as poly(isobutylene succinimide) or a surfactant such as sorbitan trioleate, sorbitan monooleate, or sorbitan monolaurate.

The circuitry may be embedded in the substrate. The circuitry may be an integrated circuit chip. The integrated circuit chip may be analog, digital, or mixed signal. An analog integrated circuit may comprise sensors, power management circuits, or operational amplifiers. A digital integrated circuit may be a microprocessor, microcontroller, memory chip, programmable device, or interface integrated circuit. The circuit may have a capacitor, diode, or resistor. Such a capacitor may be an electrolytic capacitor, a paper capacitor, a film capacitor, a ceramic capacitor, a non-polarized capacitor, or a mica capacitor. Such a diode may be a backward diode, Gunn diode, Laser diode, light emitting diode, photodiode, PIN diode, PN junction, Tunnel diode, Varactor diode, or Schottky diode. Such a resistor may be a wire wound resistor, metal film resistor, thin film resistor, network resistor, carriable resistor, light-dependent resistor, surface mount resistor, or thermistor. The circuit may create analog-to-digital conversions or digital-to-analog conversions. There may be one integrated circuit chip or a plurality thereof. The integrated circuit may process continuous signals or discrete signals. Such an integrated circuit may be comprised of only one material for all circuit elements or multiple materials for all circuit elements. A semiconductor material exhibiting either N-type or P-type conductivity may be used. Such a semiconductor may be comprised of silicon, germanium, gallium arsenide, silicon carbide, or an organic semiconductor. Such a chip may be a mini-chip. There may be one chip or a plurality thereof.

The multi-layered cover may comprise a switchable electronic-controlled capillary adhesion device (SECAD). Examples of SECAD devices are described in U.S. Pat. No. 8,998,584. The SECAD device may be capable of controlled grab and release using surface tension through capillary based adhesion that can be quickly made or broken with electronic control. In a SECAD, a liquid source may be prevented from contacting a substrate thus creating a non-adhesive detached state. In grabbing, the liquid may be pumped out of its orifice until contact is made with the substrate and a liquid bridge forms between the SECAD and the substrate. When releasing the grabbed substrate, liquid may be pumped back into the device until the liquid bridge becomes unstable and breaks. Both the attached and detached states may persist indefinitely with no additional energy added to the system and with no moving parts needed. The grab and release mechanism may be activated with an electro-osmotic pump similar to an electro-osmotic droplet switch.

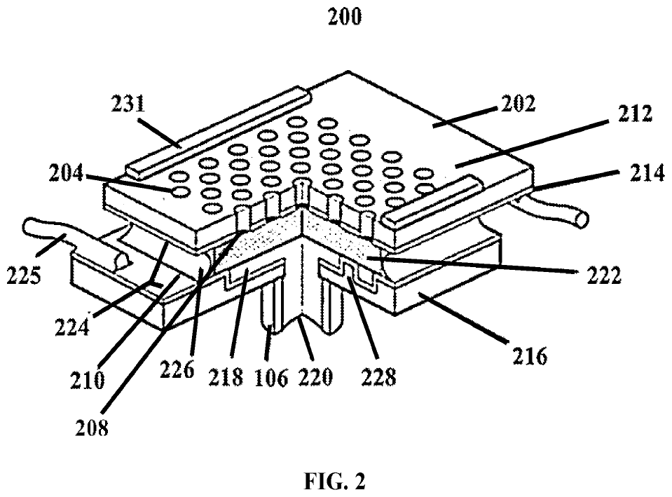

A sample SECAD device 200 is illustrated in FIG. 2. Broadly the device (or apparatus) may comprise a top layer with opposing inner and outer major surfaces, at least two fluidic thru-passageways, and a sealable fluid holder. The SECAD device 200 in FIG. 2 may include a component 202 shown as a top plate having a plurality of fluidic thru-passageways 204 each having an input end 208 and an output end 210, oriented transversely to opposing major surfaces 212 (top), 214 (bottom) of the component 202.

The top or bottom surfaces may be about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, 500 μm, 600 μm, 700 μm, 800 μm, 900 μm, or 1000 μm thick. The top of bottom surfaces may be less than about 1 μm, 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, 500 μm, 600 μm, 700 μm, 800 μm, 900 μm, or 1000 μm thick. The top of bottom surfaces may be greater than about 1 μm, 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, 500 μm, 600 μm, 700 μm, 800 μm, 900 μm, or 1000 μm thick.

The device may comprise a plurality of fluidic thru-passageways, for example, greater than or equal to about 2, 3, 5, 10, 15, 20, 25, 30, 40, 50, 60, 70, 80, 90, 100, 1000 fluidic thru-passageways, or more. The device may also include a bottom plate 216 that may comprise a fluid reservoir 218 having an inlet port 220. The fluid reservoir may be an area etched out on the inner surface of the bottom plate. Such a reservoir may have a depth of about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm. The depth may be less than about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm. The depth may be greater than about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm. An array of small pillars may stand to support the pumping material. The pumping layer may be about 0.01 millimeters (mm), 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm thick. The pumping layer may be less than about 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm thick. The pumping layer may be greater than about 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm thick.

An electro-osmotic pump 222 may be a porous layer (e.g., a glass frit in an exemplary aspect, but not limited to such material) intermediate the top and bottom plates. The diameters of the holes or pores of an electro-osmotic pump (ε) may be about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm, 600 μm, 700 μm, 800 μm, 900 μm, 1000 μm, 1500 μm, or 2000 μm. The diameters may be less than about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm, 600 μm, 700 μm, 800 μm, 900 μm, 1000 μm, 1500 μm, or 2000 μm. The diameters may be greater than about 1 micrometer (μm), 2 μm, 3 μm, 4 μm, 5 μm, 10 μm, 15 μm, 20 μm, 25 μm, 30 μm, 35 μm, 40 μm, 45 μm, 50 μm, 60 μm, 70 μm, 80 μm, 90 μm 100 μm, 110 μm, 120 μm, 130 μm, 140 μm, 150 μm, 200 μm, 250 μm, 300 μm, 350 μm, 400 μm, 450 μm, or 500 μm, 600 μm, 700 μm, 800 μm, 900 μm, 1000 μm, 1500 μm, or 2000 μm.

The number of holes in the porous layer of the electro-osmotic pump may be about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500, 2000, 2500, 3000, 3500, 4000, 4500, 5000, 6000, 7000, 8000, 9000, or 10,000. The number of holes may be less than about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500, 2000, 2500, 3000, 3500, 4000, 4500, 5000, 6000, 7000, 8000, 9000, or 10,000. The number of holes may be greater than about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500, 2000, 2500, 3000, 3500, 4000, 4500, 5000, 6000, 7000, 8000, 9000, or 10,000. The hole packing density (φ) may be sufficient for the liquid bridges to remain isolated from each other. The φ may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. The φ may be less than about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1. The φ may be greater than about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.

The hole arrays in the porous layer of the electro-osmotic pump may cover an area of about 1 millimeter² (mm²), 2 mm², 3 mm², 4 mm², 5 mm², 6 mm², 7 mm², 8 mm², 9 mm², 10 mm², 15 mm², 20 mm², 25 mm², 30 mm², 35 mm², 40 mm², 45 mm², 50 mm², 60 mm², 70 mm², 80 mm²90 mm², 100 mm², 125 mm², 150 mm², 175 mm², 200 mm², 250 mm², 300 mm², 350 mm², 400 mm², 450 mm², 500 mm², 600 mm², 700 mm², 800 mm², 900 mm², or 1000 mm². The hole arrays in the porous layer of the electro-osmotic pump may cover an area less than about 1 mm², 2 mm², 3 mm², 4 mm², 5 mm², 6 mm², 7 mm², 8 mm², 9 mm², 10 mm², 15 mm², 20 mm², 25 mm², 30 mm², 35 mm², 40 mm², 45 mm², 50 mm², 60 mm², 70 mm², 80 mm², 90 mm², 100 mm², 125 mm², 150 mm², 175 mm², 200 mm², 250 mm², 300 mm², 350 mm², 400 mm², 450 mm², 500 mm², 600 mm², 700 mm², 800 mm², 900 mm², or 1000 mm². The hole arrays in the porous layer of the electro-osmotic pump may cover an area greater than about 1 mm², 2 mm², 3 mm², 4 mm², 5 mm², 6 mm², 7 mm², 8 mm², 9 mm², 10 mm², 15 mm², 20 mm², 25 mm², 30 mm², 35 mm², 40 mm², 45 mm², 50 mm², 60 mm², 70 mm², 80 mm², 90 mm², 100 mm², 125 mm², 150 mm², 175 mm², 200 mm², 250 mm², 300 mm², 350 mm², 400 mm², 450 mm², 500 mm², 600 mm², 700 mm², 800 mm², 900 mm², or 1000 mm².

The electro-osmotic pump may have a sufficiently large zeta potential (the electrokinetic potential in colloidal dispersions) for controlling the volume of the droplets protruding from the top plate. A sufficiently large zeta potential may be important in determining the electro-osmotic pump in controlling the volume of droplets protruding from the top plate. Polar substances or polar compounds, such as untreated commercial distilled water performs well, with a zeta potential of nearly 100 millivolts (mV) although non-polar liquids or non-polar compounds (i.e., certain organics as opposed to water) may also be used to pump when properly doped, thus having an ‘effective’ zeta potential. An effective zeta potential may be about −500 mV, −450 mV, −400 mV, −350 mV, −300 mV, −250 mV, −200 mV, −150 mV, −125 mV, −100 mV, −90 mV, −80 mV, −70 mV, −60 mV, −50 mV, −45 mV, −40 mV, −35 mV, −30 mV, −25 mV, −20 mV, −15 mV, −10 mV, −9 mV, −8 mV, −7 mV, −6 mV, −5 mV, −4 mV, −3 mV, −2 mV, −1 mV, 1 mV, 2 mV, 3 mV, 4 mV, 5 mV, 6 mV, 7 mV, 8 mV, 9 mV, 10 mV, 15 mV, 20 mV, 25 mV. 30 mV, 35 mV, 40 mV, 45 mV, 50 mV, 60 mV, 70 mV, 80 mV, 90 mV, 100 mV, 125 mV, 150 mV, 200 mV, 250 mV, 300 mV, 350 mV, 400, mV, 450 mV, or 500 mV. An effective zeta potential may be less than about −500 mV, −450 mV, −400 mV, −350 mV, −300 mV, −250 mV, −200 mV, −150 mV, −125 mV, −100 mV, −90 mV, −80 mV, −70 mV, −60 mV, −50 mV, −45 mV, −40 mV, −35 mV, −30 mV, −25 mV, −20 mV, −15 mV, −10 mV, −9 mV, −8 mV, −7 mV, −6 mV, −5 mV, −4 mV, −3 mV, −2 mV, −1 mV, 1 mV, 2 mV, 3 mV, 4 mV, 5 mV, 6 mV, 7 mV, 8 mV, 9 mV, 10 mV, 15 mV, 20 mV, 25 mV. 30 mV, 35 mV, 40 mV, 45 mV, 50 mV, 60 mV, 70 mV, 80 mV, 90 mV, 100 mV, 125 mV, 150 mV, 200 mV, 250 mV, 300 mV, 350 mV, 400, mV, 450 mV, or 500 mV. An effective zeta potential may be greater than about −500 mV, −450 mV, −400 mV, −350 mV, −300 mV, −250 mV, −200 mV, −150 mV, −125 mV, −100 mV, −90 mV, −80 mV, −70 mV, −60 mV, −50 mV, −45 mV, −40 mV, −35 mV, −30 mV, −25 mV, −20 mV, −15 mV, −10 mV, −9 mV, −8 mV, −7 mV, −6 mV, −5 mV, −4 mV, −3 mV, −2 mV, −1 mV, 1 mV, 2 mV, 3 mV, 4 mV, 5 mV, 6 mV, 7 mV, 8 mV, 9 mV, 10 mV, 15 mV, 20 mV, 25 mV. 30 mV, 35 mV, 40 mV, 45 mV, 50 mV, 60 mV, 70 mV, 80 mV, 90 mV, 100 mV, 125 mV, 150 mV, 200 mV, 250 mV, 300 mV, 350 mV, 400, mV, 450 mV, or 500 mV.

As illustrated, metallized inner surfaces 224_{T,B} of the top and bottom plates 202, 216 may serve as electrodes to apply an electric field across the sandwiched middle layer for activating the electro-osmotic pump. However, this may not be the only way to activate the one or more electro-osmotic pumps. The electro-osmotic pump may be operably coupled to an electrode pair. Wire interconnects 225 to the electrodes are also shown as example. The electrode pair may comprise a top electrode and a bottom electrode. A battery operably coupled to an electro-osmotic pump may be used.

The battery may comprise an alkaline battery, atomic battery, galvanic cell, lithium battery, magnesium, magnesium-ion, mercury, zinc, nickel-cadmium, silicon-air, sodium-ion, zinc-ion, nickel oxyhydroxide, paper battery, silver-oxide, solid-state, thin film, lithium-ion, or metal-air electrochemical cell. A seal 226 around the electro-osmotic pump layer is also shown. For example, the seal can be an epoxy seal. A three-way valve 242, may provide a sealable fluid holder that is operatively coupled to the electro-osmotic pump and a fluid supply. The apparatus 200 as illustrated includes only a single electro-osmotic pump that is operatively coupled to the thru-passageways in the component; however, there may be two or more individually-addressable electro-osmotic pumps, each feeding or controlling at least two respective thru-passageways in the component. There may be one electro-osmotic pump, 2, 3, 5, 10, 15, 20, 25, 30, 40, 50, 60, 70, 80, 90, 100, 1000, or more.

Given R may be the effective pore radius of the pumping material, frits with a very fine porosity may be sufficient for pumping against small droplets at low voltage. The effective pore radius of a pumping material may be 0.01 micrometers (μm), 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm, 3.5 μm, 4 μm, 4.5 μm, 5 μm, 6 μm, 7 μm, 8 μm, 9 μm, or 10 μm. The effective pore radius of a pumping material may be less than 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm, 3.5 μm, 4 μm, 4.5 μm, 5 μm, 6 μm, 7 μm, 8 μm, 9 μm, or 10 μm. The effective pore radius of a pumping material may be greater than 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm, 3.5 μm, 4 μm, 4.5 μm, 5 μm, 6 μm, 7 μm, 8 μm, 9 μm, or 10 μm.

An effective voltage to activate the pump may be 0.1 Volts (V), 0.2 V, 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V,6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V. The effective voltage may be less than 0.1 V, 0.2 V , 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V, 6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V. The effective voltage may be greater than 0.1 V, 0.2 V , 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V, 6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V. Electro-osmotic pump materials with sufficiently fine pores, even with a reduced zeta potential, may pump against smaller droplets. Some electro-osmotic pump materials may be an aqueous electrolyte in contact with a borosilicate surface.

An important consideration may involve design and assembly care to minimize volume scavenging effects in a SECAD. Droplet-to-droplet fluid communication may travel through the flow-restricting porous pump layer. Gaps between the pump and the top plate should be substantially eliminated so that thru-passageways may be isolated from one another and directly contact the top surface of the pump. For example, exemplary devices may be fabricated with hard plastic using a traditional machine shop approach or of silicon wafers by standard photolithography techniques. Gap elimination may be achieved by fabricating the top layer of a glass frit to a flat surface to ensure good mating to the top plate. Gap elimination may also occur using rubber gaskets and the top electrode made to have identical hole patterns to the top plate and the device assembled with these layers carefully aligned. A non-limiting, exemplary order of assembly may be: top plate, gasket, electrode plate, gasket, pump surrounded along sides by gasket, electrode, gasket, bottom plate/reservoir.

The SECAD device may have a length of about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, or 20 cm. The length may be less than about 20 cm, 15 cm, 10 cm, 5 cm, 4.5 cm, 4 cm, 3.5 cm, 3 cm, 2.5 cm, 2 cm, 1.5 cm, 1.4 cm, 1.3 cm, 1.2 cm, 1.1 cm, 1 cm, 0.9 cm, 0.8 cm, 0.7 cm, 0.6 cm, 0.5 cm, or less. The length may be greater than about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, 20 cm, or more.

The SECAD device may have a width of about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, or 20 cm. The width may be less than about 20 cm, 15 cm, 10 cm, 5 cm, 4.5 cm, 4 cm, 3.5 cm, 3 cm, 2.5 cm, 2 cm, 1.5 cm, 1.4 cm, 1.3 cm, 1.2 cm, 1.1 cm, 1 cm, 0.9 cm, 0.8 cm, 0.7 cm, 0.6 cm, 0.5 cm, or less. The width may be greater than about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, 20 cm, or more.

The SECAD device may have a thickness of about 0.5 millimeters (mm), 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm. The thickness may be less than about 10 mm, 9 mm, 8 mm, 7 mm, 6 mm, 5 mm, 4.5 mm, 4 mm, 3.5 mm, 3 mm, 2.5 mm, 2 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, or less. The thickness may be greater than about 0.5 millimeters (mm), 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, or more.

The pill release device may have a length of about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, or 20 cm. The length may be less than about 20 cm, 15 cm, 10 cm, 9 cm, 8 cm, 7 cm, 6 cm, 5 cm, 4.5 cm, 4 cm, 3.5 cm, 3 cm, 2.5 cm, 2 cm, 1.5 cm, 1.4 cm, 1.3 cm, 1.2 cm, 1.1 cm, 1 cm, 0.9 cm, 0.8 cm, 0.7 cm, 0.6 cm, 0.5 cm, or less. The length may be greater than about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, 20 cm, or more.

The pill release device may have a width of about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, or 20 cm. The width may be less than about 20 cm, 15 cm, 10 cm, 9 cm, 8 cm, 7 cm, 6 cm, 5 cm, 4.5 cm, 4 cm, 3.5 cm, 3 cm, 2.5 cm, 2 cm, 1.5 cm, 1.4 cm, 1.3 cm, 1.2 cm, 1.1 cm, 1 cm, 0.9 cm, 0.8 cm, 0.7 cm, 0.6 cm, 0.5 cm, or less. The width may be greater than about 0.5 centimeters (cm), 0.6 cm, 0.7 cm, 0.8 cm, 0.9 cm, 1 cm, 1.1 cm, 1.2 cm, 1.3 cm, 1.4 cm, 1.5 cm, 2 cm, 2.5 cm, 3 cm, 3.5 cm, 4 cm, 4.5 cm, 5 cm, 10 cm, 15 cm, 20 cm, or more.

The pill release device may have a thickness of about 0.5 millimeters (mm), 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm. The thickness may be less than about 10 mm, 9 mm, 8 mm, 7 mm, 6 mm, 5 mm, 4.5 mm, 4 mm, 3.5 mm, 3 mm, 2.5 mm, 2 mm, 1.5 mm, 1.4 mm, 1.3 mm, 1.2 mm, 1.1 mm, 1 mm, 0.9 mm, 0.8 mm, 0.7 mm, 0.6 mm, 0.5 mm, or less. The thickness may be greater than about 0.5 millimeters (mm), 0.6 mm, 0.7 mm, 0.8 mm, 0.9 mm, 1 mm, 1.1 mm, 1.2 mm, 1.3 mm, 1.4 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, or more.

Grab and release may be activated by the electro-osmotic pump within a liquid-saturated porous material. The electro-osmotic pump may move liquid, efficiently against the resisting capillary pressure of the gas/liquid surfaces. A fluid mechanics-based bond using surface tension may comprise a liquid surface tension acting along the perimeter of a wetted contact area to give a force for a single contact dependent on the diameter of contact with the surface such as a liquid droplet caught between two glass histology slides. The liquid surface tension σ may act along the perimeter of a wetted contact area to give a force≈σπε for a single contact where ε=the contact diameter. The surface tension may be about 1 milliNewton per meter (mN/m), 2 mN/m, 3 mN/m, 4 mN/m, 5 mN/m, 6 mN/m, 7 mN/m, 8 mN/m, 9 mN/m, 10 mN/m, 15 mN/m, 20 mN/m, 25 mN/m, 30 mN/m, 35 mN/m, 40 mN/m, 45 mN/m, 50 mN/m, 60 mN/m, 70 mN/m, 80 mN/m, 90 mN/m, 100 mN/m, 150 mN/m, 200 mN/m, 250 mN/m, 300 mN/m, 350 mN/m, 400 mN/m, 450 mN/m, or 500 mN/m. Surface tension may be less than about 1 mN/m, 2 mN/m, 3 mN/m, 4 mN/m, 5 mN/m, 6 mN/m, 7 mN/m, 8 mN/m, 9 mN/m, 10 mN/m, 15 mN/m, 20 mN/m, 25 mN/m, 30 mN/m, 35 mN/m, 40 mN/m, 45 mN/m, 50 mN/m, 60 mN/m, 70 mN/m, 80 mN/m, 90 mN/m, 100 mN/m, 150 mN/m, 200 mN/m, 250 mN/m, 300 mN/m, 350 mN/m, 400 mN/m, 450 mN/m, or 500 mN/m. Surface tension may be greater than about 1 mN/m, 2 mN/m, 3 mN/m, 4 mN/m, 5 mN/m, 6 mN/m, 7 mN/m, 8 mN/m, 9 mN/m, 10 mN/m, 15 mN/m, 20 mN/m, 25 mN/m, 30 mN/m, 35 mN/m, 40 mN/m, 45 mN/m, 50 mN/m, 60 mN/m, 70 mN/m, 80 mN/m, 90 mN/m, 100 mN/m, 150 mN/m, 200 mN/m, 250 mN/m, 300 mN/m, 350 mN/m, 400 mN/m, 450 mN/m, or 500 mN/m.

A large number (N) of small contacts may be established each of wetted area A_{wet}. To emphasize the geometric advantage of packing perimeter into a fixed area the contact packing density may be described with ϕ=NA_{wet}/Aₙₑₜ. Using ϕ to eliminate N may yield the perimeter force as F≈Aₙₑₜ (ϕ/ε²)σε showing F ∝ 1/ε for fixed Aₙₑₜ. Taking a water/substrate contact angle into account a force of F=Nσπε sin(θ_{c}) may be expected. The contact angle may be between 0° and 360°. This amplification of the perimeter force by 1/ε may illustrate the benefit of packing a large number of small contacts into a fixed net area.

Perimeter force models may follow the general equation F=Aₘₑₐₛ where Aₘₑₐₛ is the ensemble average area of a measured subject cohort. Given the measured ensemble average of contact diameters in such a cohort, εₘₑₐₛ, F/Aₘₑₐₛ=4σ sin θ_{c}/εₘₑₐₛ. Surface tension may also generate a force via the Young-Laplace pressure equal to σπκε²/4 per contact where κ is the sum of the principal curvatures of the surface. In contrast to the perimeter force, which for bridges may only pull the substrate toward the liquid, the Young-Laplace force can either push or pull depending on κ. Adhesion strength (normal stress acting over net device area) based only on contact perimeter can be described by

$$\frac{F}{Anet} = \frac{4 ϕ σ sin ( θ c )}{ε} .$$

A successful switching between the attached and detached states may require a pump strength δ sufficient to push out and pull back liquid δ>>1 where

$$δ ≡ \frac{2 ε \vert e ζ V \vert}{β σ {R}^{2}}$$

may be a dimensionless measure of the electro-osmotic driving force against the resistance to flow by capillarity. Here e is the electric permittivity of the liquid, ζ is the zeta potential of the liquid/porous material, V is the electric potential drop across the pump, β is a scaling factor of order unity, and R is the effective pore radius of the pumping material. The electric permittivity may be a measure of the electric polarizability of a dielectric. The electric permittivity may be about 1 picofarad per meter (pF/m), 2 pF/m, 3 pF/m, 4 pF/m, 5 pF/m, 10 pF/m, 15 pF/m, 20 pF/m, 25 pF/m, 30 pF/m, 35 pF/m, 40 pF/m, 45 pF/m, 50 pF/m, 60 pF/m, 70 pF/m, 80 pF/m, 90 pF/m, 100 pF/m, 150 pF/m, 200 pF/m, 250 pF/m, 300,pF/m, 350 pF/m, 400 pF/m, 450 pF/m, 500 pF/m, 550 pF/m, 600 pF/m, 650 pF/m, 700 pF/m, 750 pF/m, 800 pF/m, 850 pF/m, 900 pF/m, 950 pF/m, 1000 pF/m, 1500 pF/m, or 2000 pF/m. The electric permittivity may be less than about 1 pF/m, 2 pF/m, 3 pF/m, 4 pF/m, 5 pF/m, 10 pF/m, 15 pF/m, 20 pF/m, 25 pF/m, 30 pF/m, 35 pF/m, 40 pF/m, 45 pF/m, 50 pF/m, 60 pF/m, 70 pF/m, 80 pF/m, 90 pF/m, 100 pF/m, 150 pF/m, 200 pF/m, 250 pF/m, 300,pF/m, 350 pF/m, 400 pF/m, 450 pF/m, 500 pF/m, 550 pF/m, 600 pF/m, 650 pF/m, 700 pF/m, 750 pF/m, 800 pF/m, 850 pF/m, 900 pF/m, 950 pF/m, 1000 pF/m, 1500 pF/m, or 2000 pF/m. The electric permittivity may be greater than about 1 pF/m, 2 pF/m, 3 pF/m, 4 pF/m, 5 pF/m, 10 pF/m, 15 pF/m, 20 pF/m, 25 pF/m, 30 pF/m, 35 pF/m, 40 pF/m, 45 pF/m, 50 pF/m, 60 pF/m, 70 pF/m, 80 pF/m, 90 pF/m, 100 pF/m, 150 pF/m, 200 pF/m, 250 pF/m, 300,pF/m, 350 pF/m, 400 pF/m, 450 pF/m, 500 pF/m, 550 pF/m, 600 pF/m, 650 pF/m, 700 pF/m, 750 pF/m, 800 pF/m, 850 pF/m, 900 pF/m, 950 pF/m, 1000 pF/m, 1500 pF/m, or 2000 pF/m. The electric potential drop across the pump may be 0.1 V, 0.2 V , 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V, 6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V. The electric potential drop across the pump may be less than 0.1 V, 0.2 V , 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V, 6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V. The electric potential drop across the pump may be greater than 0.1 V, 0.2 V , 0.3 V, 0.4 V, 0.5 V, 0.6 V, 0.7 V, 0.8 V, 0.9 V, 1 V, 1.5 V, 2 V, 2.5 V, 3 V, 3.5 V, 4 V, 4.5 V, 5 V, 6 V, 7 V, 8 V, 9 V, 10 V, 11 V, 12 V, 13 V, 14 V, 15 V, 20 V, 25 V, 30 V, 25 V, 40 V, 45 V, 50 V, 60 V, 70 V, 80 V, 90 V, or 100 V.

δ may not depend on N due to the parallel action of pressure across all holes in the top plate. The time τ to switch between the attached and detached states may be the time to move a requisite volume by the imposed flow rate of the pump. τ may be approximated by independently known parameters

$$τ = \frac{ε ϕ μ α L}{ψ \vert e ζ V \vert}$$

where α is a nondimensional spacer height, L the porous layer thickness, μ the liquid viscosity, and ψ the pump porosity. Viscosity may be 0.0001 Pascal seconds (Pa s), 0.0002 Pa s, 0.0003 Pa s, 0.0004 Pa s, 0.0005 Pa s, 0.0006 Pa s, 0.0007 Pa s, 0.0008 Pa s, 0.0009 Pa s, 0.001 Pa s, 0.002 Pa s, 0.003 Pa s, 0.004 Pa s, 0.005 Pa s, 0.006 Pa s, 0.007 Pa s, 0.008 Pa s, 0.009 Pa s, 0.010 Pa s, 0.015 Pa s, 0.02 Pa s, 0.025 Pa s, 0.03 Pa s, 0.035 Pa s, 0.04 Pa s, 0.045 Pa s, 0.05 Pa s, or 0.1 Pa s. Viscosity may be less than 0.0001 Pa s, 0.0002 Pa s, 0.0003 Pa s, 0.0004 Pa s, 0.0005 Pa s, 0.0006 Pa s, 0.0007 Pa s, 0.0008 Pa s, 0.0009 Pa s, 0.001 Pa s, 0.002 Pa s, 0.003 Pa s, 0.004 Pa s, 0.005 Pa s, 0.006 Pa s, 0.007 Pa s, 0.008 Pa s, 0.009 Pa s, 0.010 Pa s, 0.015 Pa s, 0.02 Pa s, 0.025 Pa s, 0.03 Pa s, 0.035 Pa s, 0.04 Pa s, 0.045 Pa s, 0.05 Pa s, or 0.1 Pa s. Viscosity may be greater than 0.0001 Pa s, 0.0002 Pa s, 0.0003 Pa s, 0.0004 Pa s, 0.0005 Pa s, 0.0006 Pa s, 0.0007 Pa s, 0.0008 Pa s, 0.0009 Pa s, 0.001 Pa s, 0.002 Pa s, 0.003 Pa s, 0.004 Pa s, 0.005 Pa s, 0.006 Pa s, 0.007 Pa s, 0.008 Pa s, 0.009 Pa s, 0.010 Pa s, 0.015 Pa s, 0.02 Pa s, 0.025 Pa s, 0.03 Pa s, 0.035 Pa s, 0.04 Pa s, 0.045 Pa s, 0.05 Pa s, or 0.1 Pa s.

Solid spacers may extend above the face-plane of the orifice to allow liquid bridges of the height of the spacers to form. In grabbing, liquid may be pumped out of the face pad until contact may be made with a target surface (e.g., the one or more walls of the container) and a liquid bridge may form between the device and target surface. In releasing, liquid may be pumped back into the device until the bridge becomes unstable and breaks. The spacers may assist with the release because it fixes the bridge length, enabling the liquid bridge to neck in until it pinches off and breaks. The spacer heights may be about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm. The spacer heights may be less than about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm. The spacer heights may be greater than about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 2 μm, 2.5 μm, 3 μm. Spacers may be about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 60 μm, 70 μm, 80 μm, 90 μm, or 100 μm thick. Spacers may be less than about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 60 μm, 70 μm, 80 μm, 90 μm, or 100 μm thick. Spacers may be greater than about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 60 μm, 70 μm, 80 μm, 90 μm, or 100 μm thick.

As described above or elsewhere herein, the cover may be an adhesive unit (e.g., an adhesive pad). The adhesive unit may be capable of receiving instructions from the circuitry. This circuitry may be configured to store information to direct a sequence of sealing and unsealing events of such an adhesive cover thus directing locking and unlocking a pill for release. The circuitry may be coupled to a clock or other timing device such that in response to an electrical signal from a source, such as a battery or a sensor, an adhesive cover may be sealed to its container walls for a predetermined time period. A sensor may be an infrared sensor paired with an infrared transmitter, a pressure sensor, a proximity sensor, a light sensor, or other sensor. A sensor may be a solution such as a Key Pak or Eco-Slide RX 3.0 blister solution with embedded sensing solutions manufactured by Keystone Folding Box company. These sensors and associated circuitry may track inventory of pills within the release device.

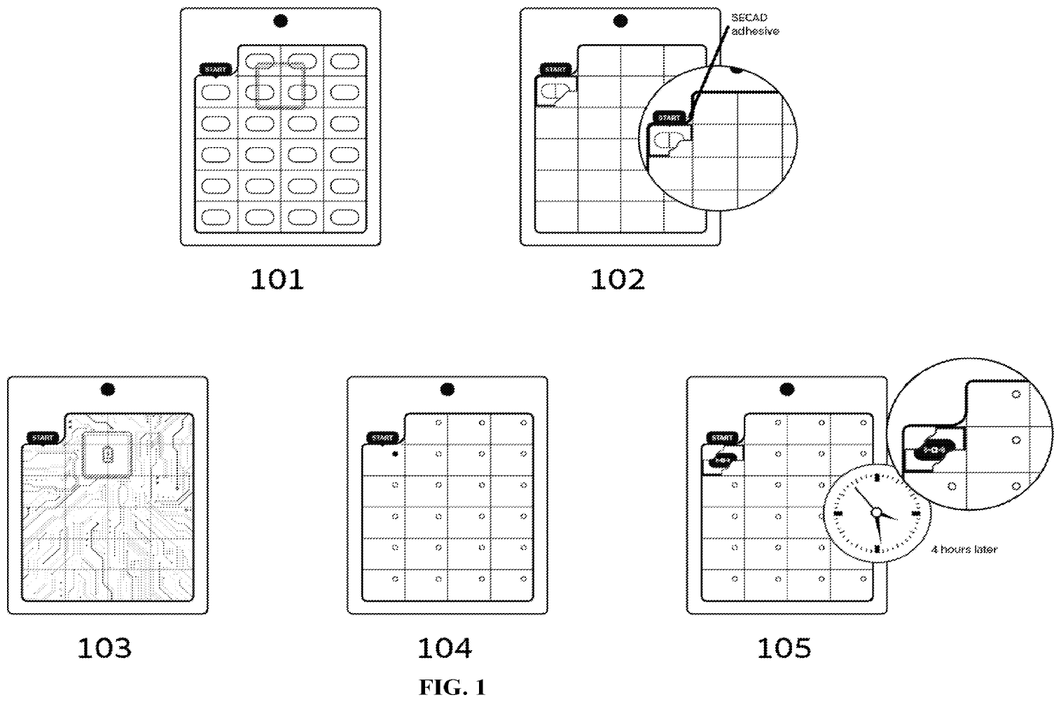

Sealing a cover for a predetermined time period may only permit an object inside the container, such as a prescription medication, to be accessed only as directed at an appropriate time interval prescribed. Such a predetermined time period may be about 15 minutes (min.), 20 min., 25 min., 30 min., 35 min., 40 min., 45 min., 50 min., 55 min., 1 hour (h), 2 h, 3 h, 4 h, 5 h, 6 h, 7 h, 8 h, 9 h, 10 h, 12 h, 14 h, 16 h, 18 h, 20 h, 22 h, 24 h, 36 h, 48 h or more than 48 h. As seen in FIG. 1, an example device of the present disclosure may be in the form of a blister pack. The device may comprise a cover which may comprise an electronically controlled adhesive pad with switchable bonding (101), such as a SECAD device. The cover can seal or unseal against the one or more walls of the container, thereby locking or unlocking the pill release device on demand. The unlocked state may act like a normal blister pack adhesive remaining closed unless a person peels off the backing (102). In this example, the blue band may be the cover comprising an adhesive pad, the backing which a patient may peel back like a normal blister pack. The electronics may be printed right onto the blister pack (103) and may enable unlocking of a given number of containers (e.g., one blister) at a time so that only the prescribed amount of medication can be taken during each time window (one unlocked “green” blister shown in 104). When the medication may be removed from the blister, a sensor may start a clock (105) (e.g. if a prescription is one pill every four hours, then four hours after the first pill may be removed from the blister pack, the second green indicator may come on and the corresponding blister may be unlocked). The patient may take then their second dose of medication. (e.g., next blister opened 4 hours later—“green” blister shown in 105). All blisters can be locked after the prescribed number of days (e.g., on the 8th day, if it is a 7-day script) even if there is unused medication.

The circuitry integrated with the device, such as a mini-chip or integrated circuit, may store at least 40 pre-programmed scripts able to direct a specific sequence of locking and unlocking events from which a health care provider may be able to choose a dosing regimen from. There may be 40 pre-programmed scripts, 50, 60, 70, 80, 90, 100, 200, 300, 400, 500, 1000, or more than 1000 pre-programmed scripts. There may be 1000, 500, 400, 300, 200, 100, 90, 80, 70, 60, 50, 40, or less than 40 pre-programmed scripts able to direct a specific sequence of locking and unlocking events. There may be pre-programmed customer modes which govern the locking and unlocking of a blister packs for safety or compliance. For example, two safety modes may be enabled in a blister pack dosing regimen. The first mode, “80%,” may be selected for a type of procedure such that 80% of patients who undergo the procedure selected may have sufficient medication to control their pain for the prescribed time period. This mode may allow normal access of medication according to a prescribed regimen, limited to the average the patient is expected to take. If additional medication is required, the patient may request additional access from their medical provider up to 80% the amount in the blister pack. A second safety mode, “24-hour lockout” may be enabled in which normal access to the full prescription is locked once a patient stops taking the medication for more than 24 hours. The patient may request additional medication up to 80% of the quantity in the blister pack to be accessed. The electronic features described herein may program approximately 40 prescription regimens, open 1-2 blisters at each dosing interval, detect that the medication was removed with a simple sensor that sits inside the blister that can signal the control system that there is either a pill in the blister or there is not a pill in the blister, and connect the blister pack to a mobile application able to implement the request system and deploy the two safety modes, “80%” and “24 hour lockout”.

A communications unit may be comprised in or operably coupled to the pill release device. The communications unit may enable a communication between the pill release device and an external device. Such a communications unit may be a communications device such as near-field communications device, a cellular modem, RFID, a Bluetooth transmitter, a USB, a WIFI transmitter, a WIMAX transmitter, or an Ethernet connection, or any other appropriate wireless communication. A communications unit may communicate to an external device through a mobile application. A mobile application may be developed which may be linked to the locking blister pack. Such an application may enable a request-approval system with a medical professional such as a pharmacist, physician, nurse practitioner, or physician assistant. Such a system may allow for prescription alterations such as requesting additional medication or accessing locked medications. This brings a check into drug access from a third-party medical professional. A mobile application may work on a cellular phone, smart phone, handheld device, personal computer, or tablet.

Also provided herein are methods for receiving or releasing a pill. The methods may comprise utilization of a pill release device as described above or elsewhere herein. For example, the pill release device may comprise a substrate comprising a container, where the container may comprise one or more walls with an opening configured to permit an object, such as a pill, to be received by or dispensed from the container, a cover configured to rest adjacent to these walls, wherein the cover and container may be configured to undergo motion relative one another; a fluid source in fluid communication with the cover, wherein said fluid source may be configured to supply a fluid to at least a portion of the cover, and where an application of the fluid from the fluid source to a portion of the cover may seal the cover against the walls; and circuitry which may be operably coupled to the fluid source, wherein the circuitry may be configured to supply one or more electrical signals to cause the fluid source to direct the fluid to or away from at least a portion of the cover, where one or more electrical signals may be sufficient to permit the cover and walls to undergo motion relative to one another, which may permit a pill to be received by or dispensed from the container. The methods may further comprise using the circuitry to supply one or more electrical signals to the fluid source. The one or more signals may cause the fluid source to direct the fluid to or away from at least a portion of the cover which may cause the cover and walls to undergo motion relative to one another which may permit a pill to be received by or dispensed from such a container. For example, a blister pack comprising a SECAD operably coupled to circuitry embedded in the backing of the blister pack may unlock a blister upon activation of the SECAD. A subject may remove the adhesive backing on this blister and remove a pill housed inside according to a dosing regimen specified by a healthcare provider.

Such a device may comprise one or a plurality of containers such that at least one pill is housed inside each container. Such a cover may have a multi-layer structure. This multi-layer structure may comprise an adhesive pad. Such an adhesive pad may provide a locking mechanism. The cover may have a thickness of less than or equal to about 5 millimeters (mm). The cover may have a thickness of less than 5 mm, 4.5 mm, 4 mm, 3.5 mm, 3 mm, 2.5 mm, 2 mm, 1.5 mm, 1 mm, or less than 0.5 mm. The cover may have a thickness greater than 5 mm, 4.5 mm, 4 mm, 3.5 mm, 3 mm, 2.5 mm, 2 mm, 1.5 mm, 1 mm, or less than 0.5 mm. The cover may comprise at least one electro-osmotic pump. The electro-osmotic pump may be part of the fluid source. The electro-osmotic pump may be external to and operably coupled to the fluid source.

The fluid source may be configured to supply a fluid which may be a polar compound. The fluid may be a doped non-polar liquid. In some cases, the cover comprises a plurality of electro-osmotic pumps. On application of the fluid from the fluid source, the multi-layered cover may be sealed or unsealed against the walls of the container. This sealing action may utilize surface tension through capillary based adhesion that can be quickly made or broken with an electronic control (e.g., an electrical pulse). In sealing, the fluid may be directed towards at least part of the cover (e.g., fluidic thru-passageways) and pumped out of the cover until contact may be made with a target surface (e.g., the one or more walls of the container) and a liquid bridge may form. When unsealing, the fluid may be pumped away from the target surface until the liquid bridge becomes unstable and breaks.

The multi-layer structure may comprise the circuitry. The adhesive unit may be capable of receiving instructions from operably coupled circuitry. This circuitry may be configured to store information able to direct a sequence of sealing and unsealing events, thus releasing a pill. This circuitry may be coupled to a clock or other timing device such that in response to an electrical signal from a source, such as a battery or a sensor, an adhesive cover may be sealed to its container walls for a predetermined time period. The sensor may be comprised in the container and may detect when a pill is removed from or dispensed into a container. This sensor may record a time when a pill is removed from or dispensed into a container.

The sensor may be in communication with the circuitry and send information to the circuitry. The circuitry may be coupled to this sensor and may supply an electrical signal within or after a predetermined time period based on the information received from the sensor or other electrical signals in the device. A predetermined time may be about 15 minutes (min), 20 min, 25 min, 30 min, 35 min, 40 min, 45 min, 50 min, 55 min, 1 hour (h), 2 h, 3 h, 4 h, 5 h, 6 h, 7 h, 8 h, 9 h, 10 h, 12 h, 14 h, 16 h, 18 h, 20 h, 22 h, 24 h, 48 h, 3 days, 4 days, 5 days, 6 days, or 7 days depending on prescription regimen. If such a predetermined time period passes, the release device may be locked even if there are pills remaining in the device.

The circuitry may send electrical signals which may permit the release device to undergo a sequence of locking and unlocking events. These locking and unlocking events may occur at a given time interval as coupled with a sensor and timing device. The time interval may be about 5 minutes (min), 10 min, 15 min, 20 min, 25 min, 30 min, 35 min, 40 min, 45 min, 50 min, 55 min, 1 hour (h), 2 h, 3 h, 4 h, 5 h, 6 h, 7 h, 8 h, 9 h, 10 h, 12 h, 14 h, 16 h, 18 h, 20 h, 22 h, 24 h, 36 h, or 48 h. If no pills are removed for approximately a 24-hour period, the release device may be locked. In cases where the release device comprises a plurality of container, the device may be locked after a given percentage of the pills are removed from the device. The given percentage of pills may be at least about 25%, 30%, 35%, 40%, 45%, 50%, 60%, 70%, 80%, 90%, or 100%. The coupled circuitry may be further configured to execute programmed scripts to direct locking and unlocking events and set time intervals of locking and unlocking events. The circuitry may be in communication with an external device such as a cellular phone, smart phone, personal computer, or tablet to accept an input comprising the scripts used. It may communicate to these external devices using an application.

### EXAMPLES

A standard blister pack may be enhanced by embedding an electrically switchable adhesive pad as a cover such that individual blisters can be locked and unlocked with a small electrical pulse. The electronically controlled adhesive pad with switchable bonding may lock and unlock on demand with the unlocked state acting like a normal blister pack cover remaining closed unless the backing is peeled off. Electronic circuitry may be printed onto the blister pack directly and may enable unlocking of one blister at a time such that only the prescribed amount of medication may be taken during each time window. A switchable electronic controlled adhesion device may be manufactured through machining techniques or through silicon wafer nanofabrication methods. This device may be capable of on-demand grab and release action with the bond strength in the grab-state approaching that of an epoxy glue. The bond strength may be sufficient such that a subject cannot access a blister. The switching time between states may be a millisecond, require low power, and the adhesive device overall may be less than 3mm thick. Maintaining the on and off states may require no additional power as the device exploits biostability such that when locked, no power is required to stay locked, when unlocked, no power is required to stay unlocked. The two states (locked and unlocked) sit at the bottom of respective energy wells, separated by an energy barrier. Switching between the locked state to the unlocked state may require a small electrical pulse and may occur via a miniature electro-osmotic pump. A small battery may be used for the locking and unlocking actions. An electro-osmotic pump may have numerous benefits such as no moving parts; low voltage/current required to switch states; fast switching (may be on the order of milliseconds); green chemistry; favorable downscaling (bond strength may be maintained even with a very small adhesive pad, as required for this application); individually addressable sub-pads; and ease of manufacture.

A coupled mini-chip may contain programmed scripts for a corresponding prescription regimen which may be enabled by a healthcare provider or a team of healthcare providers. Circuitry able to execute such a programmed script may be printed directly onto the blister pack backing materials. There may be 40, 50, 60, 70, 80, 90, 100, 200, 300, 400, 500, 1000 or more pre-programmed prescription regimens operably coupled to the blister pack. For example, a prescription may be provided by a physician for 50 milligrams (mg) of spironolactone to be taken orally once per day. A corresponding pre-programmed script for the prescription regiment may be implemented into the locking blister pack by a pharmacist to unlock one 50 mg spironolactone pill once per day for the patient to access. When a medication is removed from the blister, a sensor operably coupled to the circuitry may sense the removal of the medication and start a clock. This timing mechanism follows a programmed script which may be selected or implemented by a health care professional according to a determined dosing regimen. For example, if a prescription requires a pill be taken every four hours, then four hours after the first pill is removed from the blister pack a colored indicator may come on and the corresponding blister may unlock, and the patient may take their subsequent dose of medication. All blisters are locked after the prescribed number of days expires even if there is unused medication.

A mobile application may be developed to integrate a request system able to link to the medication release device to a healthcare provider able to alter the prescription dosing or schedule. This application may allow a request-approval system such that patients who have locked, unused medication, may request additional medication and if warranted, a healthcare provider may approve additional access.

The electronic design of this device may be configured to perform one or more functions including: (a) sending an electrical pulse to change the adhesive pad state between locked and unlocked states; (b) receiving information informing whether a pill is in a particular blister or has been removed; (c) storing a large set of prescriptions in memory so that pharmacist can select a prescription rather than input a prescription; (d) accepting input from pharmacist (pharmacists already have prescriptions in electronic form, amenable for Bluetooth connecting technology); (e) communicating with a mobile app; (f) running prescription programs which opens first blister, sets minimum time between unlocking next blister (patients may exceed that time, but never access the next pill in less than the minimum time) and the maximum number of days for prescription at which time all blisters are locked even if not all medication is used; and (g) implementing the minimum request/accept system to test whether patients can electronically request additional medication.

To perform the functions, the pill release device may comprise one or more electronic components such as (a) a small battery (which can be comprised in a multi-layered cover) to provide a pulse to unlock or lock blisters, (b) circuity (e.g., a mini-chip) that can store a plurality of scripts that may direct a specific sequence of locking and unlocking event, from which the health care provider can choose a specific dosing regimen for the blister pack, (c) a sensor (which may be comprised in or operably coupled to the container) to indicate when a pill is removed from the pack, and (d) integrated near field communications which may communicate with a mobile application.

The materials and mechanisms of this device may result in low manufacturing costs of less than 40 cents per prescription. These costs may minimize burden on the patients and may encourage large industry adoption. The methods of this device may be easily incorporated into existing pharmaceutical logistic networks.

### Computer Systems

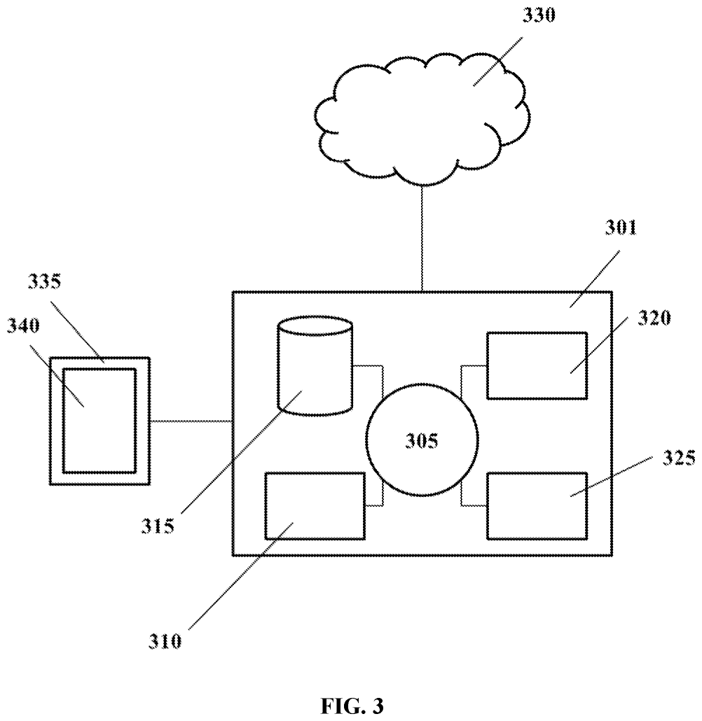

The present disclosure provides computer systems that are programmed to implement methods of the disclosure. FIG. 3 shows a computer system 301 that is programmed or otherwise configured to perform the methods described herein. The computer system 301 can regulate various aspects of the present disclosure, such as, for example, generating, storing, and/or sending instructions to the circuitry. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user (e.g., a cellular network). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, information regarding pill release, for example, number of pills removed from or dispensed into the device, time(s) when a pill is removed from or dispensed into the device. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, determine time and/or frequency at which pill are released from or dispensed into the device.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A device for receiving or release a pill, comprising:
a substrate comprising a container, which container comprises one or more walls and an opening configured to permit said pill to be received by or dispensed from said container;
a cover configured to rest adjacent to said one or more walls, wherein said cover and said container are configured to undergo motion relative one another;
a fluid source in fluid communication with said cover, wherein said fluid source is configured to supply a fluid to at least a portion of said cover, and wherein an application of said fluid from said fluid source to said at least said portion of said cover seals said cover against said one or more walls; and
circuitry operably coupled to said fluid source, wherein said circuitry is configured to supply one or more electrical signals to cause said fluid source to direct said fluid to or away from said at least said portion of said cover, which one or more electrical signals are sufficient to permit said cover and said one or more walls to undergo motion relative to one another, to thereby permit said pill to be received by or dispensed from said container.

2. The device of claim 1, wherein said substrate comprises a plurality of containers.

3. The device of claim 2, wherein said device is a blister pack.

4. (canceled)

5. The device of claim 1, wherein said cover is an adhesive pad.

6. The device of claim 1, wherein said cover has a multi-layer structure.

7. The device of claim 1, wherein said cover comprises said fluid source.

8. The device of claim 1, wherein said cover has a thickness of less than or equal to about 5 millimeters.

9. The device of claim 1, wherein said fluid comprises a polar compound.

10. The device of claim 1, wherein said circuitry is embedded in said substrate.

11. The device of claim 1, wherein said circuitry is an integrated circuit chip.

12. (canceled)

13. The device of claim 1, wherein said cover is a switchable electronic capillary adhesive device (SECAD).

14. The device of claim 13, wherein said SECAD comprises a top layer having opposing inner and outer major surfaces, at least two fluidic thru-passageways, and a sealable fluid holder.

15. The device of claim 14, wherein said fluidic thru-passageways comprise a plurality of fluidic thru-passageways.

16. The device of claim 14, wherein said at least said portion of said cover comprises one or more fluidic thru-passageways.

17. The device of claim 13, wherein said SECAD comprises at least one electro-osmotic pump operatively coupled to said fluid source.

18.-21. (canceled)

22. The device of claim 1, wherein said circuitry is further configured to store information to direct a sequence of sealing and unsealing events.

23. The device of claim 1, wherein said one or more electrical signals direct sealing of said cover against said one or more walls for a predetermined time period.

24.-43. (canceled)

44. A method for receiving or release a pill, comprising:
(a) providing a device comprising:
a substrate comprising a container, which container comprises one or more walls and an opening configured to permit said pill to be received by or dispensed from said container;
a cover configured to rest adjacent to said one or more walls, wherein said cover and said container are configured to undergo motion relative one another;
a fluid source in fluid communication with said cover, wherein said fluid source is configured to supply a fluid to at least a portion of said cover, and wherein an application of said fluid from said fluid source to said at least said portion of said cover seals said cover against said one or more walls; and
circuitry operably coupled to said fluid source, wherein said circuitry is configured to supply one or more electrical signals to cause said fluid source to direct said fluid to or away from said at least said portion of said cover, which one or more electrical signals are sufficient to permit said cover and said one or more walls to undergo motion relative to one another, to thereby permit said pill to be received by or dispensed from said container; and
(b) using said circuitry to supply said one or more electrical signals to said fluid source to direct said fluid to or away from said at least said portion of said cover, thereby causing said cover and said one or more walls to undergo motion relative to one another to permit said pill to be received by or dispensed from said container.

45.-66. (canceled)
